(19) United States
(12) Patent Application Publication
(10) Pub. No.: US 2014/0045169 A1
(43) Pub. Date: Feb. 13, 2014

(54) GLYCAN IMMOBILIZED METAL NANOPARTICLES AND USE THEREOF FOR EARLY HIV-1 DETECTION

(71) Applicants: Yasuo SUDA, Kagoshima (JP); Masanori BABA, Kagoshima (JP); Mika OKAMOTO, Kagoshima (JP)

(72) Inventors: Yasuo SUDA, Kagoshima (JP); Masanori BABA, Kagoshima (JP); Mika OKAMOTO, Kagoshima (JP)

(73) Assignee: SUDX LIFE SCIENCE CORP., Taoyuan County (TW)

(21) Appl. No.: 13/584,591

(22) Filed: Aug. 13, 2012

(51) Int. Cl. C12Q 1/70; C07H 5/06; G01N 33/569; C07H 23/00; B82Y 15/00

(52) U.S. Cl. 435/5; 536/121; 536/21; 977/773; 977/902; 977/810; 977/811

(57) ABSTRACT

Disclosed are glycan immobilized metal nanoparticles and a method used thereof for detecting HIV-1 in a saliva sample at early stages of viral infection. The method comprises the steps of: (A) providing glycan immobilized metal nanoparticles which can recognize HIV-1; (B) contacting the glycan immobilized metal nanoparticles with the saliva sample and a mixture is obtained; (C) concentrating the mixture; and (D) determining HIV-1 in the concentrated mixture by an appropriate detecting method.

### BACKGROUND OF THE INVENTION

1. Field of the Invention

The present invention relates generally to a method using glycan immobilized metal nanoparticles for detecting human immunodeficiency virus type 1 (HIV-1), and more particularly, to provide a method for detecting HIV-1 in a saliva sample at early stages of viral infection.

2. The Prior Art

Human immunodeficiency virus (HIV) is a Lenitivirus, a member of retrovirus family, which infects vital cells in the human immune system. In general, infection of HIV causes acquired immunodeficiency syndrome (AIDS), in which acquired failure of the immune system leads to life-threatening opportunistic infections and/or increases the risk of development of various viral induced cancers. AIDS was first recognized in the United States in 1981 and has been a pandemic infection since then. It has led to more than 20 million deaths until the end of 2003. Two types of HIV have been characterized: HIV-1 and HIV-2. The majority of HIV infected patients in global pertains to HIV-1 infection, and approximately 90% or more of them will be developed to AIDS in about 10-12 years after infection. However, when be infected with HIV-2, mainly confined to West Africa, there will always be no related symptoms and diseases.

Most of the surfaces of our cells are covered with glycans. It is known that glycans possess numerous functions in an organism, and one of them is being as a low-affinity receptor recognized by viruses. With the feature of recognition of glycan chains on the surface of the cells, viruses initiate the infection process of the cells. When higher animals or plants are infected by viruses, symptoms of infection will appear and transmit among the same species of animals and plants. For example, if domestic animals, agricultural crops, and ornamental animals and plants are infected and propagated between therewith, there will be a production loss thereof, so do the human been infected will damage the health. Therefore, it is crucial to have a simple and high sensitive detection method of viruses infecting animals and plants for early diagnosis, treatment or prophylaxis of infection.

HIV-1 not only exists in blood, but also in bodily fluid such as breast milk, semen, vaginal secretions, salvia, and sweat. At present, diagnosis for detecting HIV-1 in saliva is mainly by Western blotting method. For low level of HIV-1 existing in saliva, there are little anti-HIV-1 antibodies generated. Consequently, the diagnostic accuracy is low.

### SUMMARY OF THE INVENTION

The anti-HIV-1 antibodies are elicited approximately after three months of infection. During the window period, the HIV-1 was not detectable by the diagnosis method relying on the anti-HIV-1 antibodies. Accordingly, an objective of the present invention is to provide a method for concentrating little amount of HIV-1 in saliva, and make which to be capable of been analyzed by routine Western blotting or polymerase chain reaction (PCR) method.

The other objective of the present invention is to provide a diagnostic method which is non-invasive and high sensitivity for detecting HIV-1 in saliva sample.

The further objective of the present invention is to provide a ligand-conjugate in which a glycan having special structure is connected to a linker compound, and immobilized it on a metal to form a glycan immobilized metal nanoparticle. Using the character of binding of the virus to the glycan, a simple and non-invasive method for concentrating HIV-1 and detecting thereof in saliva is provided.

In order to provide a diagnosis method for detecting HIV-1 in a saliva sample at early stages of viral infection, the present invention provides a method, comprising the steps of: (A) providing glycan immobilized metal nanoparticles which can recognize HIV-1; (B) contacting the glycan immobilized metal nanoparticles with the saliva sample and a mixture is obtained; (C) concentrating the mixture; and (D) determining HIV-1 in the concentrated mixture by an appropriate detecting method; wherein each of the glycan immobilized metal nanoparticles includes a ligand-conjugate having a structure in which a linker compound is connected via an amino group thereof to a glycan having a reducing terminal; the linker compound includes, in molecules thereof, an amino group, a sulfur atom, and a hydrocarbon chain having a carbon-nitrogen bond on the main chain; the ligand-conjugate binds to a metal nanoparticle via the sulfur atom; and the glycan available for specific binding to HIV-1 is subjected to bind thereto.

According to an embodiment of the present invention, the glycan immobilized metal nanoparticle, providing the merits of stability, having equal particle size, and easy immobilization for the glycan, is capable of applying to various fields such as life science, medical diagnosis, and biotechnology. The glycan immobilized metal nanoparticle according to an embodiment of the present invention allows concentrating HIV-1 and providing safe and highly sensitive detection and identification of viruses.

Preferably, the linker compound according to an embodiment of the present invention has a structure represented by formula (I):

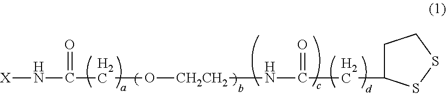

wherein a, b, c, and d are each an integer from 0 to 6; and X has a structure including an aromatic amino group in the terminal, a hydrocarbon chain having a carbon-nitrogen bond on the main chain, or has at least three branched chains, and the straight chain thereof has one chain.

Preferably, the glycan is, but not limited to, heparin, heparan sulfate, disaccharide structures that constitute heparan sulfate, chondroitin E or dextran sulfate.

Preferably, the mean particle size of the glycan immobilized metal nanoparticle is between 1 and 100 nm.

Preferably, the metal nanoparticle is, but not limited to, gold, silver, copper, aluminum, platinum, aluminum oxide, strontium titanate (SrTiO₃), lanthanum aluminate (LaAlO₃), neodymium gallate (NdGaO₃) or zirconium oxide (ZrO₂).

Preferably, the detecting method is, but not limited to, polymerase chain reaction (PCR), real time PCR, northern blotting, immunochromatography or enzyme-linked immunosorbent assay (ELISA).

The mean particle size of abovementioned glycan immobilized metal nanoparticle, in comparison with that of HIV-1, is the most appropriate one, so as to easily concentrate viruses by centrifugation and promptly detect viruses with high sensitivity.

According to the technical feature of the present invention, HIV-1 in a saliva sample which is collected by a non-invasive method is concentrated, so that a simple, fast and high sensitive diagnosis method is achieved.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a picture captured by transmission electron microscope (TEM) of the glycan immobilized metal nanoparticle which is obtained by the immobilization of dextran sulfate (DS25) having the mean molecular weight of 2,500;

FIG. 2 shows a schematic representation of the steps for the preparation of the subject for RT-PCR according to an embodiment of the present invention;

FIG. 3 shows the calibration curve by using the DNA of U1 cells; and

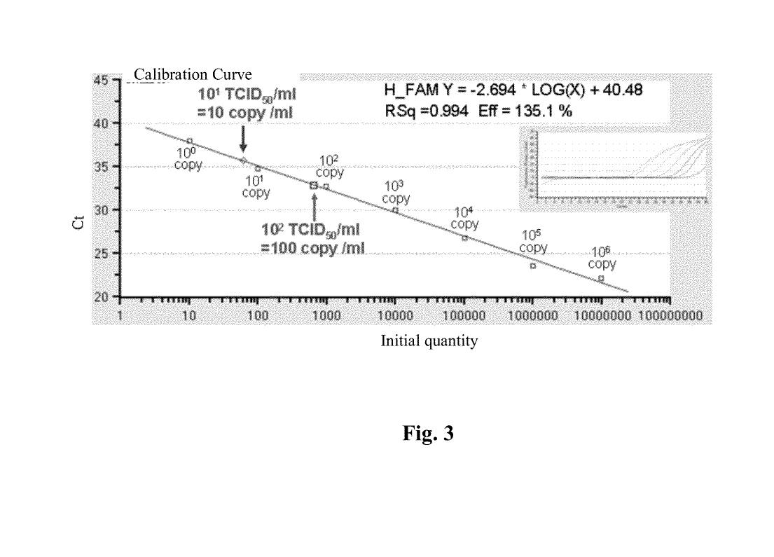

FIG. 4 is a graph showing the results of RT-PCR.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

The “glycan immobilized metal nanoparticle” according to the present invention indicates the particle that is connected by the ligand-conjugate with a metal nanoparticle via the sulfur atom.

The “ligand-conjugate” according to the present invention indicates the compound that comprises the linker compound which can bind to any kind of metal, and the glycan which can interact specifically with virus protein, wherein the linker compound is connected via the amino group to the glycan having a reducing terminal. Therefore, the ligand-conjugate is required to be the one has no specific hydrophobic interaction with proteins and related thereof.

The linker compound included in the ligand-conjugate has the sulfur atom contributed to bind with metal to form the bond of metal-S (e.g., Au—S bond) so as to link the ligand-conjugate firmly on the metal. By means of the binding of the linker compound, the glycan immobilized on the metal indirectly.

The linker compound is as represented as following formula (I) and has, in molecules thereof, an amino group and a hydrocarbon chain having a carbon-nitrogen bond on the main chain. The amino group of the linker compound is connected to the glycan having a reducing terminal and the sulfur atom thereof is bound to the metal nanoparticle, whereby the gycan chains are collected and aligned on the surface of a metal nanoparticle. Moreover, the abovementioned amino group can react with the glycan having a reducing terminal through an optimized reductive amination so as to introduce the glycan therein readily.

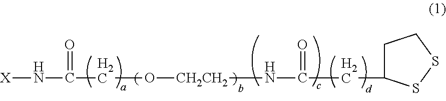

As in formula (I), a, b, c, and d are each an integer from 0 to 6; and X has a structure including an aromatic amino group in the terminal, a hydrocarbon chain having a carbon-nitrogen bond on the main chain, or has at least three branched chains, and the straight chain thereof has one chain.

The metal nanoparticle indicates a colloidal metal particle whose mean size is preferably more than 1 nm and less than 100 nm. When the particle size is less than 1 nm, it is difficult to prepare. However, when the particle size is more than 100 nm, colloidal itself may precipitate and not react with viruses properly. Further, since the size of a virus is approximately 100 nm, utilizing the particle bigger than virus may decrease the efficiency in binding to the virus. How to calculate mean particle size will be described later.

The metal may include gold, silver, copper, aluminum, platinum, aluminum oxide, strontium titanate (SrTiO₃), lanthanum aluminate (LaAlO₃), neodymium gallate (NdGaO₃), zirconium oxide (ZrO₂), etc. Gold is particularly preferable. Owing to the easy acquiring of gold, chloroauric acid (HAuCl₄) or salts thereof are preferable, and in which chloroauric acid is particularly more preferable. The method for obtaining abovementioned nanoparticle is not particularly limited. For example, it can be obtained by publicly known methods such as dissolving the chloroauric acid or salts thereof in methanol, water, or a mixture thereof, or reducing the metal ions (e.g., gold ions) to metal form (e.g., gold). An example of chloroauric acid or salts thereof may be sodium gold chloride (NaAuCl₄).

The ligand-conjugate has a structure in which the glycan having a reducing terminal is introduced to the amino group of the linker compound. In other words, the ligand-conjugate has a structure in which the linker compound is connected to the glycan having a reducing terminal via the amino group. Introduction of glycan can be carried out by a reductive amination reaction between an amino group (—NH2 group) of the linker compound and a glycan having a reducing terminal. That is, an aldehyde group (—CHO group) or a ketone group (—CRO group, R is a hydrocarbon group) of the glycan can react with the amino group included in the linker compound in an equilibrium state to produce Schiff base. By the subsequent reduction of Schiff base, the glycan can be easily introduced to the amino group of the linker compound.

When preparing the ligand-conjugate, it is preferable to mix the linker compound with the glycan at a molar ratio of 1:1 to 50:1.

The glycan having a reducing terminal comprises a monosaccharide chain, an oligosaccharide chain, or a polysaccharide chain in which the anomeric carbon atoms at their reducing terminal are not substituted. That is, the glycan having a reducing terminal is a reducing glycan. Examples of the glycan having a reducing terminal include commercially available ones, natural ones, synthetic ones, and ones prepared by the specific decomposition of commercially polysaccharide or natural polysaccharide chains.

Examples of glycan having a reducing terminal includes, but not limited to, glucose, galactose, mannose, maltose, isomaltose, lactose, panose, cellobiose, mellibiose, mannooligosaccharide, chitooligosaccharide, laminari-oligosaccaride, glucosamine, N-acetylglucosamine, glucuronic acid, heparine, heparan sulfate, chondroitin, chondroitin sulfate, dermatan sulfate, and dextran sulfate. It is preferable to choose the glycan which is recognized by viruses intended to be concentrated and can be connected to the linker compound.

The linker compound is not particular limited to a structure which contains, in its molecules, an amino group, a sulfur atom, and a hydrocarbon chain having a carbon-nitrogen bond on the main chain. Therefore, a conventional and publicly known linker compound or the linker compound developed by the inventors of the present invention may be used preferably.

The hydrocarbon chain may be such that a chain has at least one carbon-nitrogen bond; further, a part of carbon atoms and/or hydrogen atoms may be replaced with other atoms or substituent. For example, the hydrocarbon chain may be the one in which at least one carbon-carbon bond (C—C bond) is replaced with a carbon-nitrogen bond (C—N bond), or may be the one in which a part of carbon-carbon bond (C—C bond) is replaced with a carbon-nitrogen bond (C—N bond), a carbon-oxygen bond (C—O bond), an amide bond (CO—NH bond), etc. The amino group is subjected to connect the glycan, and the sulfur atom is to immobilize the ligand-conjugate onto a metal

It is preferable that the amino group is positioned at the end of the hydrocarbon chain so as to connect to the gycan readily. The amino group may be a modified one, but not limited to, which may include an amino group modified with an acetyl group, a methyl group, a formyl group, etc. or/and an aromatic amino group, wherein the aromatic amino group is particularly preferable. The amino group may be also an unmodified one. It is necessary that an amino group is not protonated under pH 3-4, which is a condition most suitable for a reductive amination reaction. Accordingly, the amino group is preferably an aromatic amino group in which conjugation with an aromatic series allows an unshared electron pair to exist on nitrogen atoms under pH 3-4.

The sulfur atom abovementioned exists in such a manner that a part of the carbons in the hydrocarbon chain are replaced with sulfur, and is capable of easily forming a metal-sulfur bond. Accordingly, it is preferable that the disulfide bond (S—S bond) or a thio group (SH group) is included in the hydrocarbon chain.

Since the glycan immobilized metal nanoparticle is obtained by mixing the ligand-conjugate with a solution containing the metal, so that the glycan can be easily immobilized.

The range of the mean particle size of the glycan immobilized metal nanoparticle is in the nano scale (more than 1 nm and less than 1 um), wherein the mean particle size which is more than 1 nm and less than 100 nm is preferable.

The “particle size” indicates the diameter of the maximum inscribed circle of a two-dimensional shape of a particle observing through the transmission electron microscope. For example, if the two-dimensional shape of a particle is substantially circular, the “particle size” indicates the diameter of the circle. If it is substantially ellipse, the “particle size” indicates the minor axis of the ellipse. Besides, if it is substantially square, the “particle size” indicates a side of the square. If it is substantially rectangle, the “particle size” indicates a short side of the rectangle. The “mean particle size” indicates a mean value of particle sizes of a plurality of particles. The mean particle size described herein is determined by measuring the mean value of the particle sizes of 20 particles.

According to an embodiment of the present invention, the test sample is mixed with the glycan immobilized metal nanoparticle and then determined by centrifugation. If there exists HIV-1, HIV-1 would bind to the glycan chains of the glycan immobilized metal nanoparticle. The test sample is not particularly limited as long as it can contact and react with the glycan immobilized metal nanoparticle. Examples of the test sample may include saliva, nasal mucosa, blood, etc. Those body fluid previous described can be used directly as specimen or may be used in the form by adding MEM culture medium or normal saline. Alternatively, it is available to mix the glycan immobilized metal nanoparticle with water, normal saline, or phosphate buffer, whereby the viruses in the test sample can contact with the glycan immobilized metal nanoparticle.

Since the virus can recognize glycan, the glycan capable of been recognized by virus is introduced to the glycan immobilized metal nanoparticle, thus the viruses suspected to be contained in the test sample will specifically bind to the glycan chains thereof.

Viruses intended to be concentrated according to the present invention are HIV-1.

Examples of glycan chains that can be specifically recognized by HIV-1 include, but not limited to, heparine, heparan sulfate, disaccharide structures that constitute heparan sulfate, chondroitin E, and dextran sulfate. By selecting one of these glycan chains, the glycan immobilized metal nanoparticle is then prepared. Subsequently, contacting the glycan immobilized metal nanoparticle with the test sample, if the viruses are contained in the test sample, they will specifically bind to the glycan chains.

Typically, the HIV-1 in the test sample is approximately 0.1 to 10 copies/ml, but the concentration thereof can be readily and promptly concentrated and increased according to the concentration method of the present invention and subsequent isolation by centrifugation (e.g. 10,000 g), such that detection of HIV-1 by RT-PCR can be carried out owing to the sufficient template.

By means of centrifugation, viruses bound to the glycan immobilized metal nanoparticle are precipitated. The method for recovering the virus DNA from the precipitate is not particularly limited, and the publicly known methods are also available. For examples, washing the precipitate with sterilized water or distilled water first, and then suspending the precipitate therein and heating it to the temperature above 90° C., preferably 100° C., and the virus DNA is finally recovered from the supernatant.

The mixture abovementioned is obtained by contacting the test sample with the glycan immobilized metal nanoparticle. If the HIV-1 exists in the test sample, the HIV-1 will recognize and bind to the glycan. The “contacting the test sample with the glycan immobilized metal nanoparticle” previously described indicates that mixing the test sample with the solution prepared by adding water, normal saline, or phosphate buffer solution to the glycan immobilized metal nanoparticle. The mixture can be agitated by pipetting so as to make complete binding of virus with glycan and shorten the binding time needed.

The “recognize” as mentioned above indicates the “binding” of surface protein of a virus with a glycan chain via a sugar-binding site (sugar chain recognition site) thereon. Examples of the binding form include binding by hydrogen bonding, ion bonding, electrostatic interaction and van der Waals force.

The method for confirming the result of the concentration of viruses in accordance with the present invention is not particularly limited, and a conventional and publicly known method may be used. Examples of the method for confirming the collected viruses include polymerase chain reaction (PCR), real time PCR, northern blotting, immunochromatography and enzyme-linked immunosorbent assay (ELISA). In particular, real time PCR is preferable since it allows promptly determining the DNA quantity. By real time PCR method, a difference is calculated by subtracting a Ct (Threshold Cycle) value after concentration from a Ct value before concentration. If the difference calculated through the concentration according to the present invention is almost equal to that calculated through the concentration by centrifugation, an object of the present invention may be considered to be solved. When the Ct value is more than 35, the amplification of DNA comes almost from the contaminate nucleic acid and not of viruses. Whether the DNA of viruses is amplified or not can be determined by analyzing the temperature-melting curve (Tm curve) of the RT-PCR amplifying after 40 cycles.

The present invention is not limited to the description of the embodiments above, but may be altered by a skilled person within the scope of the claims. An embodiment based on a proper combination of technical means disclosed in different embodiments is encompassed in the technical scope of the present invention.

### EXAMPLES

The following more specifically explains the present invention with reference to these Examples. It should be noted that the present invention is not limited to these Examples.

### Example 1

Concentration of HIV-1 in saliva sample and high sensitively detecting of HIV-1 according to the present invention

20 nM NaAuCl₄.2H₂O (7.956 mg/ml), 50 nM NaBH₄ (7.6 mg/ml), and 5 nM DS25 ligand-conjugate (12.5 mg/ml) were first prepared. 2.08 ml of 20 nM NaAuCl₄.2H₂O was mixed with 33.45 ml of ultra pure water, and then 4.08 ml of 50 nM NaBH₄ was added and stirred for 5 sec. Afterward 1.52 ml of 5 nM DS25 ligand-conjugate was added therein and stirred for 30 sec. The mixture was subsequently stirred in dark for 30 min at room temperature. Following stirring, dialysis with membrane in molecular weight of 1000 cut was proceeded (in the order of ultra pure water, PBS, ultra pure water). Finally, membrane in molecular weight of 3500 cut was provided for further dialysis (with ultra pure water). The solution after dialysis was aliquoted to 3 tubes each with 15 ml and then dried by freeze drying. The dried pellet was dissolved in ultra pure water to a concentration of 10 mg/ml and then centrifuged at 4200 rpm for 30 min. The concentration of the precipitate was adjusted to OD value of 0.5 under UV 530 nm so that the Solution I, glycan immobilized metal nanoparticle (hereinafter referred to as “DS25 immobilized metal nanoparticle”) with mean particle size value of 5 nm, was obtained.

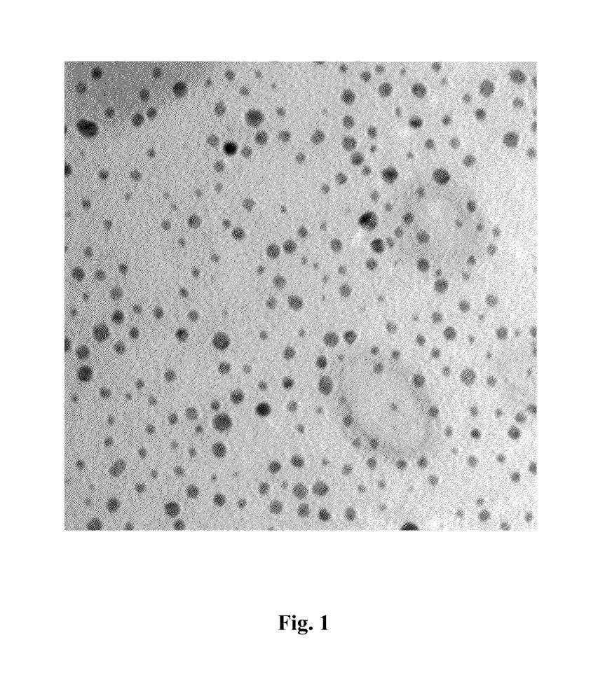

FIG. 1 is a picture captured by transmission electron microscope of the glycan immobilized metal nanoparticle which is formed by the immobilization of dextran sulfate (DS25) having the mean molecular weight of 2500. The DS25 was added with acetic acid, so was the N, N-dimethylacetoamide solution (hereinafter referred to as “DMAC solution”), which is a linker compound represented as general formula (I), wherein a=0, b=0, and c=0. The molar ratio of DS25 added with acetic acid to DMAC solution added with acetic acid and to NaBH₃CN is 1:1:10. All of the solutions mentioned above were mixed and stirred at 37° C. for 3 days.

The saliva sample was diluted with PBS to half of the concentration and adjusted to a volume of 500 μl, in which 10 μl was taken as a control. Solution II was obtained by mixing 10 μl of Solution I with 490 μl diluted saliva sample, which was stirred at a room temperature and then stayed for 30 min.

Afterwards Solution II was centrifuged at 10,000 g for 10 min, and the supernatant and the precipitate were collected. The precipitate was added with 10 μl of ultra pure water and then heated at 100° C. for 5 min. Following heating, the solution was subjected to centrifuge again at 10,000 g for 10 min, whereby the supernatant was obtained.

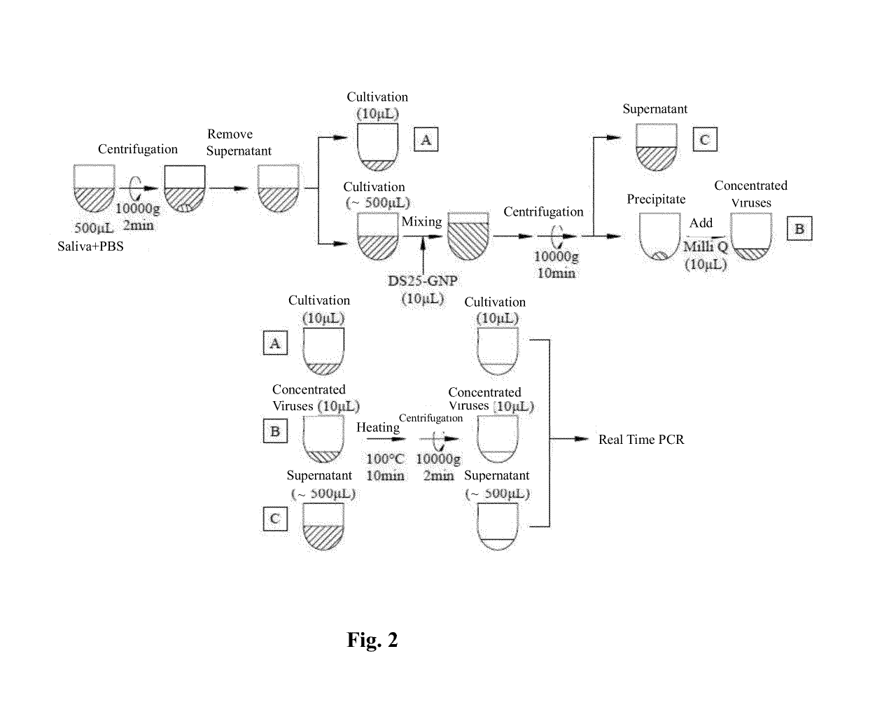

FIG. 2 shows the steps of preparation of Solution II and the supernatant employed for RT-PCR.

2 μl of the supernatant was added to PCR reagents and 20 μl thereof was taken and subjected to RT-PCR. The device used was Thermal Cycler Dice Real Time System TP800 (product number TP800, manufactured by TAKARABIO). The reagents used were Takara One Step SYBR® (Prime Script® RT-PCR Kit II) and SYBR Green was used as a fluorochrome. Primers used herein were 581F (5′-TGGTAACTAGAGTCCCCTCAGACC-3′, SEQ ID NO: 1) and 620T ('5-AGCTCCTCTGGTTTCCCTTTC-3′, SEQ ID NO: 2). The calibration curve was made by the DNA integrated in host cell with 2 copies of HIV-1 DNA to measure the absolute quantity of HIV-1 DNA in the test sample. Conditions for RT-PCR were such that reverse transcription reaction was carried out at 45° C. for 2 min, initial thermal degeneration process was carried out at 95° C. for 1 min, and PCR cycle was set for 40 cycles with each cycle under 95° C. for 1 sec, 60° C. for 1 sec, and 72° C. for 5 sec.

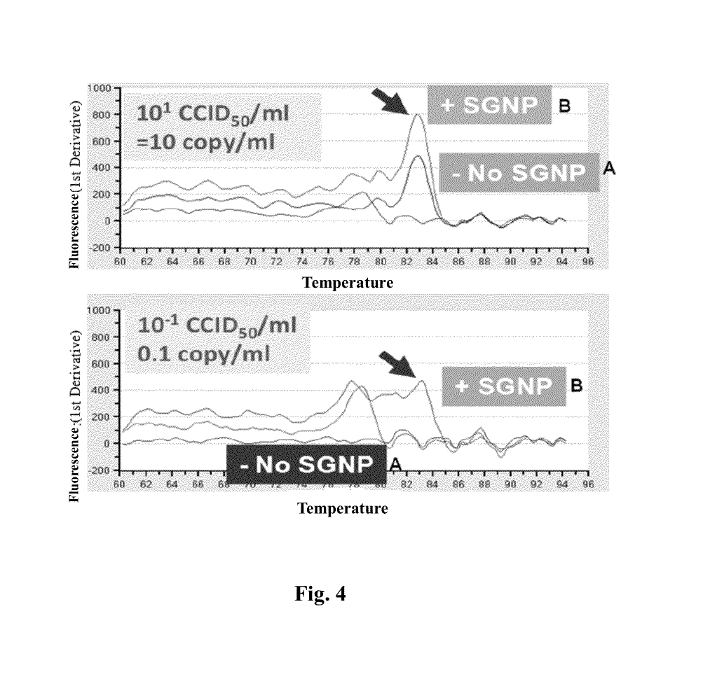

According to the results of RT-PCR, the HIV-1 contained in salvia sample reacted with the DS25 immobilized metal nanoparticle and formed the precipitate (step A in FIG. 2), comparing to the result of the sample not been added with the DS25 immobilized metal nanoparticle (step B in FIG. 2), shows that the RNA of which was actually identified by analysis of Tm curve (FIG. 4).

Using the calibration curve made from the DNA of U1 cell, the HIN-1 contained in diluted saliva sample can be detected. 10 and 100 CCID50/ml respectively indicates 10 and 100 copies/ml. By the method according to the present invention, the efficiency of HIV-1 DNA determination is notably increased by concentrating HIV-1 DNA with DS25 immobilized metal nanoparticle when the amount of HIV-1 DNA is in the range of 0.1 to 10 copies/ml (FIG. 4), namely even only been little amount, HIV-1 DNA can also be detected by means of concentration of test sample.

Furthermore, by means of heparin (with the mean molecular weight of 17,500) immobilized metal nanoparticle and detecting the HIV-1 in saliva sample with the same method described above could also identify the presence of HIV-1 even through the concentration of HIV-1 DNA thereof is as low as 0.1 copies/ml (FIG. 4).

Utilizing the heparin immobilized metal nanoparticle to concentrate HIV-1, even though the concentration of HIV-1 is as low as 0.1 copies/ml, it can be detected through RT-PCR. Thus, it is a superior method for detecting HIV-1 in saliva sample by the combination of the concentration of the heparin immobilized metal nanoparticle and RT-PCR method. The saliva sample can be obtained easily and non-invasively, and always subjected to the diagnosis of infection, ulcer, heart disease, etc. The HIV-1 contained in saliva is about 20% of that in blood, such that the detection method according to the present invention provides higher sensitivity than that of blood sample by means of combining the concentration of the heparin immobilized metal nanoparticle and RT-PCR method. Although the kit for saliva sample testing can be acquired in the market, the kit using anti-HIV-1 antibody in Western blotting has the shortcoming that the HIV-1 cannot be detected during three months window period in which there were no anti-HIV-1 antibody appeared. However, by concentrating the HIV-1 through combination of the concentration of the heparin immobilized metal nanoparticle and RT-PCR method, HIV-1 can be concentrated and detected in saliva sample even by Western blotting method. In general, HIV-1 exists in blood, breast milk, semen, and vaginal secretions, and transmits through them. But in saliva, if there was no bleeding, it is very difficult to detect HIV-1 therein. Accordingly, the method according to the present invention can provide an easy, effective and safe diagnosis method at early stages of HIV-1 infection.

As embodiments mentioned above, the present invention providing a detection method of HIV-1 in saliva sample at early stages of HIV-1 infection is subject to the utility demand. Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for detecting HIV-1 in a saliva sample at early stages of infection, comprising the steps of:
(A) providing glycan immobilized metal nanoparticles which can recognize HIV-1;
(B) contacting the glycan immobilized metal nanoparticles with the saliva sample and a mixture is obtained;
(C) concentrating the mixture; and
(D) determining HIV-1 in the concentrated mixture by an appropriate detecting method;
wherein each of the glycan immobilized metal nanoparticles includes a ligand-conjugate having a structure in which a linker compound is connected via an amino group thereof to a glycan having a reducing terminal; the linker compound includes, in molecules thereof, an amino group, a sulfur atom, and a hydrocarbon chain having a carbon-nitrogen bond on the main chain; the ligand-conjugate binds to a metal nanoparticle via the sulfur atom; and the glycan available for specific binding to HIV-1 is subjected to bind thereto.

2. The method according to claim 1, wherein the linker compound has the formula (I): 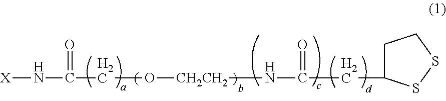
wherein a, b, c, and d are each an integer from 0 to 6; and X has a structure including an aromatic amino group in the terminal, a hydrocarbon chain having a carbon-nitrogen bond on the main chain, or has at least three branched chains, and the straight chain thereof has one chain.

3. The method according to claim 1, wherein the glycan is heparin, heparan sulfate, disaccharide structures that constitute heparan sulfate, chondroitin E, or dextran sulfate.

4. The method according to claim 1, wherein the mean particle size of the glycan immobilized metal nanoparticle is between 1 and 100 nm.

5. The method according to claim 1, wherein the metal nanoparticle is gold, silver, copper, aluminum, platinum, aluminum oxide, strontium titanate (SrTiO₃), lanthanum aluminate (LaAlO₃), neodymium gallate (NdGaO₃), or zirconium oxide (ZrO₂).

6. The method according to claim 1, wherein the detecting method is polymerase chain reaction (PCR), real time PCR, Northern blotting, immunochromatography, or enzyme-linked immunosorbent assay (ELISA).

7. A glycan immobilized metal nanoparticle which is capable of recognizing HIV-1, comprising a ligand-conjugate having a structure in which a linker compound is connected via an amino group thereof to a glycan having a reducing terminal; the linker compound includes, in molecules thereof, an amino group, a sulfur atom, and a hydrocarbon chain having a carbon-nitrogen bond on the main chain; the ligand-conjugate binds to a metal nanoparticle via the sulfur atom; and the glycan available for specific binding to HIV-1 is subjected to bind thereto.

8. The glycan immobilized metal nanoparticle according to claim 7, wherein the linker compound has the formula (I): 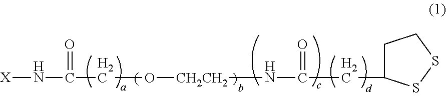
wherein the a, b, c and d are each an integer from 0 to 6; and X has a structure including an aromatic amino group in the terminal, a hydrocarbon chain having a carbon-nitrogen bond on the main chain, or has at least three branched chains, and the straight chain thereof has one chain.

9. The glycan immobilized metal nanoparticle according to claim 7, wherein the glycan is heparin, heparan sulfate, disaccharide structures that constitute heparan sulfate, chondroitin E, or dextran sulfate.

10. The glycan immobilized metal nanoparticle according to claim 7, wherein the mean particle size of the glycan immobilized metal nanoparticle is between 1 and 100 nm.

11. The glycan immobilized metal nanoparticle according to claim 7, wherein the metal nanoparticle is gold, silver, copper, aluminum, platinum, aluminum oxide, strontium titanate (SrTiO₃), lanthanum aluminate (LaAlO₃), neodymium gallate (NdGaO₃), or zirconium oxide (ZrO₂).


SEQUENCE LISTING (2 sequences)

SEQ ID NO: 1 - DNA, 23 bp, Artificial Sequence
  primer 581F for RT-PCR
tggtaactagagtccctcagacc

SEQ ID NO: 2 - DNA, 21 bp, Artificial Sequence
  primer 620T for RT-PCR
agctcctctggtttccctttc